(19) United States
(12) Patent Application Publication
(10) Pub. No.: US 2021/0100274 A1
(43) Pub. Date: Apr. 8, 2021

(54) Nutritional composition for use in the prevention of dry skin

(71) Applicant: N.V. Nutricia, Zoetermeer (NL)

(72) Inventors: Marieke Abrahamse-Berkeveld, Utrecht (NL); Dennis Stanley Acton, Utrecht (NL); Stefanie Schoen, Utrecht (NL)

(73) Assignee: N.V. Nutricia, Zoetermeer (NL)

(21) Appl. No.: 17/044,394

(22) PCT Filed: Apr. 3, 2019
(86) PCT No.: PCT/EP2019/058378

§ 371 Date: Oct. 1, 2020

(30) Foreign Application Priority Data
Apr. 3, 2018 (EP) 18165420.3
Oct. 23, 2018 (EP) 18202108.9

(51) Int. Cl. A23L 33/00; A23L 33/18; A23L 33/115; A23L 29/30; A23L 33/26

(52) U.S. Cl. CPC A23L 33/40; A23L 33/18; A23L 33/26; A23L 29/30; A23L 33/115

(57) ABSTRACT

The invention relates to a nutritional composition for infants, in particular infant formula, follow on formula or growing up milk. The invention further relates to the prevention of dry skin in an infant.

### FIELD OF THE INVENTION

The invention relates to a nutritional composition for infants, in particular infant formula, follow on formula or growing up milk. The invention further relates to the prevention of dry skin in infants.

### BACKGROUND OF THE INVENTION

Human milk is the uncontested gold standard concerning infant nutrition. However, in some cases breastfeeding is inadequate or unsuccessful for medical reasons or not available because of a choice not to breastfeed. For such situations infant or follow on formulas have been developed.

Commercial infant formulas are commonly used today to provide supplemental or sole source of nutrition early in life. These formulas comprise a range of nutrients to meet the nutritional needs of the growing infant, and typically include fat, carbohydrate, protein, vitamins, minerals, and other nutrients helpful for optimal infant growth and development. Commercial infant formulas are designed to mimic, as closely as possible, the composition and function of human milk.

Lipids in infant formulas and human milk are crucial in fulfilling nutritional needs for infants as the lipid fraction of milk is providing almost half of the caloric intake of infants. The lipid globules in human milk have a volume-based mode diameter of 3-5 μm and are enveloped by a tri-layered membrane mainly made of phospholipids, membrane-specific proteins, and cholesterol, which structural complexity originates from their synthesis and secretion by the epithelial cells of the mammary gland.

The lipid moiety in current infant formulas is derived from vegetable oils and is present as lipid globules with a volume-based mode diameter of approximately 0.5 μm and with proteins as the main emulsifier (at the surface of lipid globules).

Over the past decades, substantial improvements in nutritional (lipid) quality of infant formulas have been realized. An infant formula production process was developed that resulted in larger lipid globules (having a volume based mode diameter of at least 1 μm) having a thin interface of milk phospholipids and other polar lipids, (glyco)proteins and cholesterol (NUTURIS®). These changes in lipid globule characteristics were found to alter in vitro lipid digestion kinetics, the postprandial lipid response in adult men, to prevent excessive fat accumulation and adverse metabolic outcomes in a murine nutritional programming model and to improve specific cognitive behaviours in mice.

Xeroderma or xerodermia, derived from the Greek words for “dry skin”, is a condition of the skin, which in most cases can safely be treated with emollients or moisturizers. Xeroderma occurs most commonly on the scalp, lower legs, arms, hands, the knuckles, the sides of the abdomen, and thighs. Symptoms most associated with xeroderma are scaling (the visible peeling of the outer skin layer), itching, and skin cracking. In the context of the present invention, xeroderma is considered to be unrelated to a disorder or skin disorder. Prevention of xeroderma is therefore considered to be of a non-therapeutic nature.

Kim et al. (An Analysis of Dietary Intakes and Plasma Biochemical Indices in Female College Students by Skin Types, Korean J Community Nutr. 1999 March; 4(1):20-29) investigated the nutrient intakes and plasma biochemical indices in 68 female college students according to their skin types. The intakes of energy and fats in oily skin group were significantly higher (p<0.05) than those of the dry skin group, but vitamin C intake in the mixed skin group was significantly higher (p<0.05) than those of the dry skin group, but vitamin C intake in the mixed skin group was significantly lower (p<0.05) than that in other skin types. The intakes of other nutrients were not significantly different among skin types. The researchers conclude that overall results indicate that dietary intake pattern may influence skin type and thereby some blood biochemical indices can be different by skin types.

Cosgrove et al. (Dietary nutrient intakes and skin-aging appearance among middle-aged American women, Am J Clin Nutr, October 2007, vol. 86, no. 4, 1225-1231) evaluated the associations between nutrient intakes and skin-aging appearance. Their conclusion was that higher intakes of vitamin C and linoleic acid and lower intakes of fats and carbohydrates are associated with better skin-aging appearance. Promoting healthy dietary behaviours may have additional benefit for skin appearance in addition to other health outcomes in the population.

### SUMMARY OF THE INVENTION

A study on the growth and safety of an experimental infant formula during 3-4 months intervention was conducted in healthy, term infants compared to a standard formula and a breast-fed reference. In a randomized, controlled, multi-centre, double-blinded, prospective clinical trial, infants were enrolled and assigned to receive one of two infant formulae until 17 weeks of age: 1) TEST infant formula, an infant formula comprising lipid in the form of large lipid globules comprising a coating comprising phospholipids or 2) CONTROL infant formula, with standard, smaller, lipid globules coated with protein. The composition of the formulas was similar in energy and macronutrient composition.

During the intervention period the number of adverse events was monitored and the inventors of the present invention have surprisingly found that the experimental TEST infant formula had an effect on the occurrence of dry skin. More in particular, the number of subjects with one or more events of dry skin was significantly lower in the TEST group compared to the CONTROL group.

The prevention of dry skin in an infant is considered to be non-therapeutic. The present invention therefore pertains to a non-therapeutic method of preventing dry skin in an infant, comprising feeding said infant a nutritional composition, selected from infant formula, follow on formula and growing up milk, wherein said composition comprises lipid, protein and digestible carbohydrates, wherein the lipid is in the form of lipid globules, wherein
a. the lipid globules have a mode diameter based on volume of at least 1 μm; and/or
b. at least 45 vol. % of the lipid globules has a diameter of 2 to 12 μm, and/or
c. the lipid comprises at least 0.5 wt. % phospholipid based on total lipid and the lipid globules have a coating comprising phospholipid,
   and wherein the nutritional composition is not human milk.

### DETAILED DESCRIPTION OF THE INVENTION

For some jurisdictions, the invention can also be worded as the use of lipid, protein and digestible carbohydrates, wherein the lipid is in the form of lipid globules, wherein
a. the lipid globules have a mode diameter based on volume of at least 1 μm; and/or
b. at least 45 vol. % of the lipid globules has a diameter of 2 to 12 μm, and/or
c. the lipid comprises at least 0.5 wt. % phospholipid based on total lipid and the lipid globules have a coating comprising phospholipid,
   in the manufacture of a nutritional composition, selected from infant formula, follow on formula and growing up milk, for preventing dry skin in an infant, and wherein the nutritional composition is not human milk.

For some jurisdictions, the invention can also be worded as a nutritional composition, selected from infant formula, follow on formula and growing up milk, for use in the prevention of dry skin in an infant, wherein said composition comprises lipid, protein and digestible carbohydrates, wherein the lipid is in the form of lipid globules, wherein
a. the lipid globules have a mode diameter based on volume of at least 1 μm; and/or
b. at least 45 vol. % of the lipid globules has a diameter of 2 to 12 μm, and/or
c. the lipid comprises at least 0.5 wt. % phospholipid based on total lipid and the lipid globules have a coating comprising phospholipid,
   and wherein the nutritional composition is not human milk.

For some jurisdictions, the invention can also be worded as a method for preventing dry skin in an infant, said method comprising feeding said infant a nutritional composition, selected from infant formula, follow on formula and growing up milk, said nutritional composition comprising lipid, protein and digestible carbohydrates, wherein the lipid is in the form of lipid globules, wherein
a. the lipid globules have a mode diameter based on volume of at least 1 μm; and/or
b. at least 45 vol. % of the lipid globules has a diameter of 2 to 12 μm, and/or
c. the lipid comprises at least 0.5 wt. % phospholipid based on total lipid and the lipid globules have a coating comprising phospholipid,
   and wherein the nutritional composition is not human milk.

The invention can also be worded as the use of a nutritional composition, selected from infant formula, follow on formula and growing up milk, which comprises lipid, protein and digestible carbohydrates, wherein the lipid is in the form of lipid globules, wherein
a. the lipid globules have a mode diameter based on volume of at least 1 μm; and/or
b. at least 45 vol. % of the lipid globules has a diameter of 2 to 12 μm, and/or
c. the lipid comprises at least 0.5 wt. % phospholipid based on total lipid and the lipid globules have a coating comprising phospholipid,
   and which is not human milk, for prevention of dry skin in an infant.

In the context of the present invention, ‘dry skin’ is not dry skin that can be associated with a skin disease or seen as a symptom of a disorder, such as for example dry skin as a symptom of atopic dermatitis. Hence, in one embodiment according to the present invention, prevention of dry skin in an infant is not prevention of atopic dermatitis in an infant.

The term ‘dry skin’ as used herein, refers to xeroderma or xerodermia as described herein before. In the context of the present invention, xeroderma or xerodermia refers to peeling or cracking of the skin that is not associated with a skin disease, such as atopic dermatitis. Hence, in one embodiment according to the present invention, prevention of xeroderma or xerodermia in an infant is not prevention of atopic dermatitis in an infant.

In the context of the present invention, the nutritional composition is not native cow's milk.

### Lipid Globule Size

According to the present invention, lipid is present in the nutritional composition in the form of lipid globules. When the nutritional composition is in liquid form, these lipid globules are emulsified in the aqueous phase. Alternatively, when the nutritional composition is in powder form, the lipid globules are present in the powder and the powder is suitable for reconstitution with water or another food grade aqueous phase. The lipid globules comprise a core and a surface.

The lipid globules in the nutritional composition preferably have mode diameter, based on volume, of at least 1.0 μm, more preferably at least 3.0 μm, and most preferably at least 4.0 μm. Preferably, the lipid globules have a mode diameter, based on volume, between 1.0 and 10 μm, more preferably between 2.0 and 8.0 μm, even more preferably between 3.0 and 7.0 μm, and most preferably between 4.0 μm and 6.0 μm.

Alternatively, or preferably in addition, the size distribution of the lipid globules is preferably in such a way that at least 45 volume % (vol. %), preferably at least 55 vol. %, even more preferably at least 65 vol. %, and most preferably at least 75 vol. % of the lipid globules have a diameter between 2 and 12 μm. In a preferred embodiment, at least 45 vol. %, preferably at least 55 vol. %, more preferably at least 65 vol. %, and most preferably at least 75 vol. % of the lipid globules have a diameter between 2 and 10 μm. In a more preferred embodiment, at least 45 vol. %, more preferably at least 55 vol. %, yet even more preferably at least 65 vol. %, and most preferably at least 75 vol. % of the lipid globules have a diameter between 4 and 10 μm. Preferably less than 5 vol. % of the lipid globules have a diameter above 12 μm.

Standard infant formulae, follow on formulae or growing up milks have lipid globules with a mode diameter, based on volume, of below 0.5 μm. It was found that the presence of large lipid globules with a mode diameter, based on volume, of at least 1 μm, or that a significant part, based on volume, of the lipid globules has a diameter between 2 to 12 μm, prevents the development of dry skin in an infant.

The percentage of lipid globules is based on volume of total lipid. The mode diameter relates to the diameter which is the most present based on volume of total lipid, or the peak value in a graphic representation, having on the X-as the diameter and on the Y-as the volume (%).

The volume of the lipid globule and its size distribution can suitably be determined using a particle size analyzer such as a Mastersizer (Malvern Instruments, Malvern, UK), for example by the method described in Michalski et al, 2001, Lait 81: 787-796.

### Phospholipid

Preferably the lipid in the nutritional composition comprises at least 0.5 wt. % phospholipid based on total lipid. Preferably, the lipid globules in the nutritional composition have a coating comprising phospholipid. Hence in a preferred embodiment, the lipid in the nutritional composition comprises at least 0.5 wt. % phospholipid based on total lipid and the lipid globules in the nutritional composition have a coating comprising phospholipid.

Preferably the nutritional composition comprises 0.5 to 20 wt. % phospholipid based on total lipid, more preferably 0.5 to 10 wt. %, more preferably 0.75 to 8 wt. %, even more preferably 1.0 to 8 wt. % even more preferably 1.5 to 5 wt. % phospholipid based on total lipid.

Phospholipids are amphipathic of nature and include glycerophospholipids and sphingomyelin. By ‘coating’ is meant that the outer surface layer of the lipid globules comprises phospholipid, whereas phospholipid is virtually absent in the core of the lipid globule. The presence of phospholipid in the nutritional composition was found to advantageously prevent the development of dry skin in infants. A suitable way to determine whether phospholipid is located on the surface of lipid globules is confocal laser scanning microscopy or transmission electron microscopy; see for instance Gallier et al. (A novel infant milk formula concept: Mimicking the human milk fat globule structure, Colloids and Surfaces B: Biointerfaces 136 (2015) 329-339).

The nutritional composition preferably comprises glycerophospholipids. Examples of glycerophospholipids are phosphatidylcholine (PC), phosphatidylserine (PS), phosphatidylethanolamine (PE), phosphatidylinositol (PI) and phosphatidylglycerol (PG). Preferably the nutritional composition comprises one or more of PC, PS, PI and PE, more preferably the nutritional composition comprises at least PC.

The nutritional composition preferably comprises sphingomyelin. Sphingomyelins have a phosphorylcholine or phosphorylethanolamine molecule esterified to the 1-hydroxy group of a ceramide. They are classified as phospholipid as well as sphingolipid, but are not classified as a glycerophospholipid nor as a glycosphingolipid. Preferably the nutritional composition comprises 0.05 to 10 wt. % sphingomyelin based on total lipid, more preferably 0.1 to 5 wt. %, even more preferably 0.2 to 2 wt. %. Preferably the nutritional composition comprises at least 5 wt. %, more preferably 5 to 40 wt. % sphingomyelin based on total phospholipid, more preferably 10 to 35 wt. %, even more preferably 15 to 35 wt. %, based on total phospholipid.

The nutritional composition preferably comprises glycosphingolipids. The term glycosphingolipids in the present context particularly refers to glycolipids with an amino alcohol sphingosine. The sphingosine backbone is O-linked to a charged head-group such as ethanolamine, serine or choline backbone. The backbone is also amide linked to a fatty acyl group. Glycosphingolipids are ceramides with one or more sugar residues joined in a beta-glycosidic linkage at the 1-hydroxyl position, and include gangliosides. Preferably the nutritional composition contains gangliosides, more preferably at least one ganglioside selected from the group consisting of GM3 and GD3. Preferably the nutritional composition comprises 0.1 to 10 wt. % glycosphingolipids based on total lipid, more preferably 0.5 to 5 wt. %, even more preferably 2 to 4 wt. %, based on total lipid.

The nutritional composition preferably comprises cholesterol. The nutritional composition preferably comprises at least 0.005 wt. % cholesterol based on total lipid, more preferably at least 0.02 wt. %, more preferably at least 0.05 wt. %., even more preferably at least 0.1 wt. % cholesterol based on total lipid. Preferably the amount of cholesterol does not exceed 10 wt. % based on total lipid, more preferably does not exceed 5 wt. %, even more preferably does not exceed 1 wt. % based on total lipid in the nutritional composition.

Preferred sources for providing the phospholipid, glycosphingolipid and/or cholesterol are egg lipids, milk fat, buttermilk fat and butter serum fat (such as beta serum fat). A preferred source for phospholipid, particularly PC, is soy lecithin and/or sunflower lecithin.

The nutritional composition preferably comprises phospholipid derived from milk. Preferably the nutritional composition comprises phospholipid and glycosphingolipid derived from milk. Preferably also cholesterol is obtained from milk. The nutritional composition preferably comprises phospholipid, glycosphingolipid and/or cholesterol from milk of cows, mares, sheep, goats, buffalos, horses and camels. More preferably the nutritional composition comprises phospholipid, glycosphingolipid and/or cholesterol from cow's milk.

Phospholipid derived from milk includes preferably phospholipid that is isolated from milk lipid, cream lipid, cream serum lipid, butter serum lipid (beta serum lipid), whey lipid, cheese lipid and/or buttermilk lipid. Buttermilk lipid is typically obtained during the manufacture of buttermilk. Butter serum lipid or beta serum lipid is typically obtained during the manufacture of anhydrous milk fat from butter. Preferably the phospholipid, glycosphingolipid and/or cholesterol is obtained from milk cream. Suitable commercially available sources for phospholipid from milk are BAEF, SM2, SM3 and SM4 powder of Corman, Salibra of Glanbia, and LacProdan MFGM-10 or PL20 from Arla.

The use of phospholipid from milk fat advantageously comprises the use of milk fat globule membranes, which are more reminiscent to the situation in human milk. The concomitant use of phospholipid derived from milk and triglycerides derived from vegetable lipids therefore enables the manufacture of coated lipid globules with a coating more similar to human milk, while at the same time providing an optimal fatty acid profile.

Preferably the phospholipid is derived from milk lipid, more preferably from milk fat globule membrane (MFGM). Preferably the phospholipid is derived from cow's milk lipid, more preferably from cow's MFGM.

Preferably the nutritional composition comprises phospholipid and glycosphingolipid and in a preferred embodiment the weight ratio of phospholipid:glycosphingolipid is from 2:1 to 12:1, more preferably from 2:1 to 10:1 and even more preferably 2:1 to 5:1.

Methods for obtaining lipid globules with an increased size and/or coating with phospholipid are for example disclosed in WO 2010/0027258 and WO 2010/0027259.

Lipid in the form of large lipid globules and the presence of phospholipid, preferably phospholipid comprised in a coating of the lipid globules, prevents the development of dry skin in infants.

Hence in a preferred embodiment, the nutritional composition comprises lipid wherein the lipid is in the form of lipid globules, which have a mode diameter, based on volume, of at least 1 μm and/or at least 45 vol. % of the lipid globules has a diameter of 2 to 12 μm, and the lipid comprises at least 0.5 wt. % phospholipid based on total lipid and the lipid globules have a coating comprising phospholipid.

### Lipid

The nutritional composition according to the present use comprises lipid. Lipid in the present invention comprises one or more selected from the group consisting of triglycerides, polar lipids (such as phospholipids, cholesterol, glycolipids, sphingomyelin), free fatty acids, monoglycerides and diglycerides.

The lipids provide preferably 30 to 60% of the total calories of the nutritional composition. More preferably the nutritional composition comprises lipid providing 35 to 55% of the total calories, even more preferably the nutritional composition comprises lipids providing 40 to 50% of the total calories. The lipids are preferably present in an amount of 4 to 6 g per 100 kcal. When in liquid form, e.g. as a ready-to-feed liquid, the nutritional composition preferably comprises 2.1 to 6.5 g lipids per 100 ml, more preferably 3.0 to 4.0 g per 100 ml. Based on dry weight the nutritional composition preferably comprises 10 to 50 wt. %, more preferably 12.5 to 40 wt. % lipids, even more preferably 19 to 30 wt. % lipids.

The lipid preferably comprises vegetable lipids. The presence of vegetable lipids advantageously enables an optimal fatty acid profile, high in polyunsaturated fatty acids and/or more reminiscent to human milk fat. Lipids from non-human mammalian milk alone, e.g. cow's milk, do not provide an optimal fatty acid profile. The amount of essential fatty acids is too low in non-human mammalian milk.

Preferably the nutritional composition comprises at least one, preferably at least two vegetable lipid sources selected from the group consisting of linseed oil (flaxseed oil), rape seed oil (such as colza oil, low erucic acid rape seed oil and canola oil), sunflower oil, high oleic sunflower oil, safflower oil, high oleic safflower oil, olive oil, coconut oil, palm oil and palm kernel oil.

In one preferred embodiment, the nutritional composition comprises 5 to 100 wt. % vegetable lipids based on total lipids, more preferably 10 to 95 wt. %, more preferably 20 to 80 wt. %, even more preferably 25 to 75 wt. %, most preferably 40 to 60 wt. %. It is noted therefore that the nutritional composition also may comprise non-vegetable lipids. Non-vegetable lipids may include mammalian milk fat, mammalian milk derived lipid as a preferred source of phospholipid, and fish, marine and/or microbial oils as source of LC-PUFA.

### Palmitic Acid (PA) at Sn-2 Position of Triglyceride

Triglycerides are the major fraction of the lipids in the nutritional composition. Triglycerides comprise a glycerol moiety to which, via ester bonds, three fatty acid residues are attached, which may be the same or different, and which are generally chosen from saturated and unsaturated fatty acids containing 4 to 26 carbon atoms. Such triglycerides may differ in the fatty acid residues that are present and/or may differ in the respective position(s) of the fatty acid residues to the glycerol backbone (e.g. in the sn-1, sn-2 and/or sn-3 position). Preferably the nutritional composition comprises at least 70 wt. %, more preferably at least 80 wt. %, more preferably at least 85 wt. % triglycerides based on total lipids, even more preferably at least 90 wt. % triglycerides based on total lipids, even more preferably at least 95 wt. % triglycerides based on total lipids.

Further improved prevention of dry skin in infants was observed when the lipid component had an increased amount of palmitic acid (PA) acid located at the sn-2 position in a triglyceride, based on total PA.

Lipids that can be used to enhance the amount of PA located at the sn-2 position in triglycerides based on total PA are commercially available—e.g. from Loders Croklaan under the name Betapol™ and/or can be prepared in a manner known per se, for instance as described in EP 0698078 and/or EP 0758846. Another suitable source is InFat™ of Enzymotec. In case these lipids are obtained by trans- or interesterification of vegetable triglycerides, these sources are in the context of the present invention regarded as vegetable lipids.

A preferred source for triglycerides to enhance PA at the sn-2 or beta position in a triglyceride is non-human animal fat, more preferably non-human mammalian milk fat, even more preferably cow's milk fat. Preferably non-human mammalian milk fat, in particular cow's milk fat, is used in the form of anhydrous milk fat, butter oil, butter fat or butter. Preferably, the source of the milk fat is in a homogenous fat phase, such as butter oil or anhydrous milk fat, and not in the form of oil in water emulsion such as cream, since the lipid globules of the present invention can be more easily prepared during the manufacture of the nutritional composition for the use according to the present invention, when the lipid is added to the aqueous phase as homogenous fat phase, upon which the mixture is treated to form an emulsion.

Preferably the amount of the source of lipid, comprising triglyceride with an increased amount of palmitic acid residues in the sn-2 position of a triglyceride, that is comprised in the lipid of the nutritional composition, is between 10 and 99.5 wt. %, more preferably between 15 and 85 wt. % based on total lipid, more preferably between 20 and 75 wt. %, more preferably between 25 and 65 wt. %, even more preferably between 30 and 60 wt. % based on total lipid. Such source of lipid is preferably mammalian milk fat, more preferably such source of lipids is mammalian milk fat which is selected from butter, butter fat, butter oil or anhydrous milk fat. Preferably the nutritional composition comprises mammalian milk fat between 5 and 95 wt. %, more preferably between 20 and 80 wt. % based on total lipid, more preferably between 25 and 75 wt. %, even more preferably between 40 and 60 wt. % based on total lipid.

In a particularly preferred embodiment, the lipid in the nutritional composition comprises:
a. 30 to 90 wt. % vegetable fat based on total lipid, and
b. 10 to 70 wt. % mammalian milk fat based on total lipid.

More preferably, the lipid in the nutritional composition comprises:
a. 35 to 75 wt. % vegetable fat based on total lipid, and
b. 25 to 65 wt. % mammalian milk fat based on total lipid.

Most preferably, the lipid in the nutritional composition comprises:
a. 40 to 60 wt. % vegetable fat based on total lipid, and
b. 40 to 60 wt. % mammalian milk fat based on total lipid.

The sources of the lipid in the nutritional composition are preferably chosen such that the amount of palmitic acid (PA) that is present in the total lipid of the nutritional composition is at least 10 wt. % based on total fatty acids in the total lipid, preferably at least 15 wt. %. Preferably the amount of PA that is present in the total lipid is below 30 wt. % based on total fatty acids. More preferably the amount of PA that is present in the lipid is from 15 to 24 wt. % based on total fatty acids in the total lipid, even more preferably from 15 to 19 wt. %, even more preferably from 16 to 19 wt. %.

The lipids in the nutritional composition are preferably chosen such that, based on the total PA present in the lipid, at least 15 wt. %, preferably at least 20 wt. %, more preferably at least 25 wt. %, more preferably at least 30 wt. % PA is in the sn-2 or beta position in a triglyceride. Preferably the amount of PA in the sn-2 position in a triglyceride is not more than 45 wt. %, preferably not more than 40 wt. % based on total PA present in the lipid. Preferably the amount of PA in the sn-2 position in a triglyceride is from 25 to 40 wt. % based on total PA present in the total lipid.

Lipid in the form of large lipid globules and comprising a coating comprising phospholipid, together with an optimal fatty acid composition of the nutritional composition and the presence of an enhanced amount of sn-2 palmitic acid, showed a further improvement of the prevention of dry skin in an infant.

### Fatty Acid Composition

Herein LA refers to linoleic acid and/or acyl chain (18:2 n6); ALA refers to alpha-linolenic acid and/or acyl chain (18:3 n3); SFA relates to saturated fatty acids and/or acyl chains, MUFA relates to mono-unsaturated fatty acid and/or acyl chains, PUFA refers to polyunsaturated fatty acids and/or acyl chains with 2 or more unsaturated bonds; LC-PUFA refers to long chain polyunsaturated fatty acids and/or acyl chains comprising at least 20 carbon atoms in the fatty acyl chain and with 2 or more unsaturated bonds; DHA refers to docosahexaenoic acid and/or acyl chain (22:6, n3); EPA refers to eicosapentaenoic acid and/or acyl chain (20:5 n3); ARA refers to arachidonic acid and/or acyl chain (20:4 n6); DPA refers to docosapentaenoic acid and/or acyl chain (22:5 n3). PA relates to palmitic acid and/or acyl chains (C16:0). Medium chain fatty acids (MCFA) refer to fatty acids and/or acyl chains with a chain length of 6, 8 or 10 carbon atoms. n3 or omega 3 PUFA refers to polyunsaturated fatty acids and/or acyl chains with 2 or more unsaturated bonds and with an unsaturated bond at the third carbon atom from the methyl end of the fatty acyl chain, n6 or omega 6 PUFA refers to polyunsaturated fatty acids and/or acyl chains with 2 or more unsaturated bonds and with an unsaturated bond at the sixth carbon atom from the methyl end of the fatty acyl chain. BA refers to butyric acid (4:0).

The nutritional composition according to the present use preferably comprises LA. LA is an n6 PUFA and the precursor of n6 LC-PUFA and is an essential fatty acid as it cannot be synthesized by the human body. LA preferably is present in a sufficient amount in order to promote a healthy growth and development, yet in an amount as low as possible to prevent negative, competitive, effects on the formation of n3 PUFA and a too high n6/n3 ratio. The nutritional composition therefore preferably comprises less than 25 wt. %, more preferably less than 20 wt. %, more preferably less than 15 wt. % LA based on total fatty acids. The nutritional composition preferably comprises at least 5 wt. % LA based on fatty acids, preferably at least 7.5 wt. %, more preferably at least 10 wt. % based on total fatty acids.

The nutritional composition preferably comprises ALA. ALA is a n3 PUFA and the precursor of n3 LC-PUFA and is an essential fatty acid as it cannot be synthesized by the human body. Preferably ALA is present in a sufficient amount to promote a healthy growth and development of the infant. The nutritional composition therefore preferably comprises at least 0.5 wt. %, more preferably at least 1.0 wt. %, more preferably the nutritional composition comprises at least 1.5 wt. %, even more preferably at least 2.0 wt. % ALA based on total fatty acids. Preferably the nutritional composition comprises less than 10 wt. % ALA, more preferably less than 5.0 wt. % based on total fatty acids.

The weight ratio LA/ALA preferably is well balanced in order to ensure an optimal n6/n3 PUFA, n6/n3 LC PUFA and DHA/ARA ratio in the cellular membranes. Therefore, the nutritional composition preferably comprises a weight ratio of LA/ALA from 2 to 20, more preferably from 3 to 15, more preferably from 5 to 12, more preferably from 5 to 10. Preferably the n6 PUFA/n3 PUFA weight ratio is from 3 to 20, more preferably from 3 to 15, more preferably from 5 to 12, more preferably from 5 to 10.

Preferably, the nutritional composition comprises n3 LC-PUFA, such as EPA, DPA and/or DHA, more preferably DHA. As the conversion of ALA to DHA may be less efficient in infants, preferably both ALA and DHA are present in the nutritional composition. Preferably the nutritional composition comprises at least 0.05 wt. %, preferably at least 0.1 wt. %, more preferably at least 0.2 wt. %, of DHA based on total fatty acids. Preferably the nutritional composition comprises not more than 2.0, preferably not more than 1.0 wt. %, of DHA based on total fatty acids.

The nutritional composition preferably comprises ARA. Preferably the nutritional composition comprises at least 0.05 wt. %, preferably at least 0.1 wt. %, more preferably at least 0.2 wt. %, of ARA based on total fatty acids. As the group of n6 fatty acids, especially arachidonic acid (ARA) counteracts the group of n3 fatty acids, especially DHA, the nutritional composition preferably comprises relatively low amounts of ARA. Preferably the nutritional composition comprises not more than 2.0 wt. %, preferably not more than 1.0 wt. %, of ARA based on total fatty acids. Preferably the weight ratio between DHA and ARA is between 1/4 to 4/1, more preferably between 1/2 to 2/1, more preferably between 0.6 and 1.5.

### Digestible Carbohydrates

The nutritional composition comprises digestible carbohydrates. The digestible carbohydrates preferably provide 30 to 80% of the total calories of the nutritional composition. Preferably the digestible carbohydrates provide 40 to 60% of the total calories. Based on calories the nutritional composition preferably comprises of 5 to 20 g of digestible carbohydrates per 100 kcal, more preferably 7.5 to 15 g. When in liquid form, e.g. as a ready-to-feed liquid, the nutritional composition preferably comprises 3.0 to 30 g digestible carbohydrate per 100 ml, more preferably 6.0 to 20, even more preferably 7.0 to 10.0 g per 100 ml. Based on dry weight the nutritional composition preferably comprises 20 to 80 wt. %, more preferably 40 to 65 wt. % digestible carbohydrates.

Preferred digestible carbohydrate sources are lactose, glucose, sucrose, fructose, galactose, maltose, starch and maltodextrin. Lactose is the main digestible carbohydrate present in human milk. Lactose advantageously has a low glycemic index. The nutritional composition preferably comprises lactose. The nutritional composition preferably comprises digestible carbohydrate, wherein at least 35 wt. %, more preferably at least 50 wt. %, more preferably at least 75 wt. %, even more preferably at least 90 wt. %, most preferably at least 95 wt. % of the digestible carbohydrate is lactose. Based on dry weight the nutritional composition preferably comprises at least 25 wt. % lactose, preferably at least 40 wt. %.

### Protein

The nutritional composition comprises protein. The protein preferably provides 5 to 15% of the total calories. Preferably the nutritional composition comprises protein that provides 6 to 12% of the total calories. More preferably protein is present in the nutritional composition below 3.5 gram per 100 kcal, more preferably the nutritional composition comprises between 1.8 and 2.1 g protein per 100 kcal, even more preferably between 1.85 and 2.0 g protein per 100 kcal. A low protein concentration advantageously is closer to human milk as human milk comprises a lower amount of protein based on total calories than cow's milk. The protein concentration in a nutritional composition is determined by the sum of protein, peptides and free amino acids. Based on dry weight the nutritional composition preferably comprises less than 12 wt. % protein, more preferably between 9.6 and 12 wt. %, even more preferably between 10 and 11 wt. %. Based on a ready-to-drink liquid product the nutritional composition preferably comprises less than 1.5 g protein per 100 ml, more preferably between 1.2 and 1.5 g, even more preferably between 1.25 and 1.35 g.

The source of the protein should be selected in such a way that the minimum requirements for essential amino acid content are met and satisfactory growth is ensured. Hence protein sources based on cows' milk proteins such as whey, casein and mixtures thereof and proteins based on soy, potato or pea are preferred. In case whey proteins are used, the protein source is preferably based on acid whey or sweet whey, whey protein isolate or mixtures thereof. Preferably the nutritional composition comprises at least 3 wt. % casein based on dry weight. Preferably the casein is intact and/or non-hydrolyzed. For the present invention protein includes peptides and free amino acids.

### Non Digestible Carbohydrates

In one embodiment the nutritional composition preferably comprises non-digestible oligosaccharides. Preferably the nutritional composition comprises non-digestible oligosaccharides with a degree of polymerization (DP) between 2 and 250, more preferably 3 and 60. The non-digestible oligosaccharides advantageously further prevent the development of dry skin in an infant.

Preferably the nutritional composition comprises fructo-oligosaccharides, galacto-oligosaccharides and/or galacturonic acid oligosaccharides, more preferably fructo-oligosaccharides and/or galacto-oligosaccharides, even more preferably galacto-oligosaccharides, most preferably transgalacto-oligosaccharides. In a preferred embodiment the nutritional composition comprises a mixture of galacto-oligosaccharides and fructo-oligosaccharides, more preferably transgalacto-oligosaccharides and fructo-oligosaccharides. Suitable non-digestible oligosaccharides are for example Vivinal®GOS (FrieslandCampina DOMO), Raftilin®HP or Raftilose® (Orafti).

Preferably, the nutritional composition comprises 80 mg to 2 g non-digestible oligosaccharides per 100 ml, more preferably 150 mg to 1.50 g, even more preferably 300 mg to 1 g per 100 ml. Based on dry weight, the nutritional composition preferably comprises 0.25 wt. % to 20 wt. %, more preferably 0.5 wt. % to 10 wt. %, even more preferably 1.5 wt. % to 7.5 wt. %. A lower amount of non-digestible oligosaccharides will be less effective in preventing dry skin in infants, whereas a too high amount will result in side-effects of bloating and abdominal discomfort.

### Formula

The use according to the present invention requires the administration of an infant formula, a follow on formula or a growing up milk. This means that the composition that is administered is not human milk. It also means that the composition that is administered is not native cow's milk or native milk from another mammal. Alternatively the terms as used herein, “infant formula” or “follow on formula” or “growing up milk” means that it concerns a composition that is artificially made or in other words that it is synthetic. Hence in one embodiment the nutritional composition that is administered is an artificial infant formula or an artificial follow on formula or an artificial growing up milk or a synthetic infant formula or a synthetic follow on formula or a synthetic growing up milk.

In the present context, infant formula refers to nutritional compositions, artificially made, intended for infants of 0 to about 4 to 6 months of age and are intended as a substitute for human milk. Typically infant formulae are suitable to be used as sole source of nutrition. Such formulae are also known as starter formula. Formula for infants starting with at 4 to 6 months of life to 12 months of life are intended to be supplementary feedings to infants that start weaning on other foods. Such formulae are also known as follow on formulae. Infant formulae and follow on formulae are subject to strict regulations, for example for the EU Commission Directive 2006/141/EC. In the present context, growing up milk refers to nutritional compositions, artificially made, intended for infants of 12 months to 36 months, which are intended to be supplementary feedings to infants.

The nutritional composition is preferably an infant formula or a follow on formula. More preferably the nutritional composition is an infant formula.

The nutritional composition is preferably an infant formula or follow on formula and preferably comprises 3 to 7 g lipid/100 kcal, preferably 4 to 6 g lipid/100 kcal, more preferably 4.5 to 5.5 g lipid/100 kcal, preferably comprises 1.7 to 5 g protein/100 kcal, preferably 1.8 to 3.5 g protein/100 kcal, more preferably 1.8 to 2.1 g protein/100 kcal, more preferably 1.8 to 2.0 g protein/100 kcal and preferably comprises 5 to 20 g digestible carbohydrate/100 kcal, preferably 6 to 16 g digestible carbohydrate/100 kcal, more preferably 10 to 15 g digestible carbohydrate/100 kcal.

Preferably the nutritional composition is an infant formula or follow on formula, when ready to drink has an energy density of 60 kcal to 75 kcal/100 ml, more preferably 60 to 70 kcal/100 ml. This density ensures an optimal balance between hydration and caloric intake.

In one embodiment, the nutritional composition is a powder. Suitably, the nutritional composition is in a powdered form, which can be reconstituted with water or other food grade aqueous liquid, to form a ready-to drink liquid, or is in a liquid concentrate form that should be diluted with water to a ready-to-drink liquid. It was found that lipid globules maintained their size and coating when reconstituted.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### EXAMPLES

### Example 1

This clinical study was conducted in 17 study centres in four countries including The Netherlands, Belgium, France and Singapore.

### Subjects and Study Design

Healthy term infants, with a gestational age between 37 weeks and 42 weeks, postnatal age ≤35 days, and either fully formula-fed or fully breastfed were eligible for participation. The study was designed as a randomized, double-blind, controlled, prospective, multi-country, equivalence trial. After enrolment, formula-fed infants (n=223) were randomly assigned to receive the TEST formula (n=115) or the CONTROL formula (n=108) ad libitum using region (Europe/Asia), sex (male/female) and infants' age at randomization (≤14 days/>14 days) as strata. For analysis of safety parameter ((serious) adverse events), 3 subjects randomised to TEST formula and 5 subjects randomised to CONTROL formula, were excluded as they did not receive any study product. Breastfed infants (n=88) served as a reference group and were eligible if the mother intended to breastfeed exclusively for at least 13 weeks. During the study, infants were fully formula-fed or fully breast-fed.

Infants had an enrolment (baseline) visit ≤35 days of age, followed by visits at 5, 8, 13 and 17 weeks of age. In case the baseline was conducted within 2 weeks before the visit at 5 weeks, parents only were invited again to the study centre at their infants age of 8 weeks and beyond. During the study, (serious) adverse events were documented by the investigators at each visit. In addition, 4 phone calls (at 6, 11, 15 and 19 weeks of age) were conducted to record any new or changes in adverse events. Adverse events were followed-up by the investigator until they had abated or until a stable situation had been reached.

### Study Infant Formulas

The two study infant formulas used in this study were complete standard cow's milk-based infant formulas that comprised per 100 ml reconstituted formula 66 kcal, 1.3 g protein (intact protein with a casein/whey ratio of 40/60), 7.3 g digestible carbohydrates (mainly lactose), 3.4 g fat and 0.8 g short chain galacto-oligosaccharides (source Vivinal® GOS) and long chain fructo-oligosaccharides (source Raftilin HP®) in a 9/1 w/w ratio, and minerals, vitamins trace elements and other micronutrients as known in the art and in compliance with directives for infant formula. The formula was provided as a powder with the instruction to reconstitute with water. About 13.6 g powder was to be reconstituted to 100 ml water to obtain the reconstituted infant formula. The two study formulas only differed in lipid sources used and in lipid globule size (Table 1). The fatty acid composition was very similar between the CONTROL formula and the TEST formula, in saturated, mono unsaturated and poly unsaturated fatty acids, and in n3- and n6-PUFA content.

### Control Formula

The fat component comprised mainly a vegetable fat (blend of palm oil, low erucic acid rape seed oil, coconut oil, high oleic sunflower oil, sunflower oil) and about 1.5 wt. % of an LC-PUFA premix (fish oil and microbial oil). No milk derived phospholipid was added.

### Test Formula

The fat component comprised of about 50 wt. % vegetable fat (blend of low erucic acid rape seed oil, coconut oil, high oleic sunflower oil, sunflower oil), about 44 wt. % bovine anhydrous milk fat, 1.5 wt. % LC-PUFA containing oil (fish oil and microbial oil), about 3.6 wt. % milk fat derived from buttermilk rich in milk phospholipid or milk fat globule membranes (milk phospholipid is about 1.5 wt. % based on total lipid). The lipid droplets in the TEST formula had a volume-based mode diameter of 5.6 μm and an interface predominantly composed of milk phospholipids following a production process as described in WO 2013/135739.

TABLE 1
Composition of the study formulas (per 100 ml)

| | CONTROL formula | | TEST formula | |
|---|---|---|---|---|
| Per 100 ml | Weight | | Weight | |
| Total lipid | 3.4 | g | 3.4 | g |
| Linoleic acid | 447 | mg | 447 | mg |
| Alpha linolenic acid | 82 | mg | 83 | mg |
| Arachidonic acid | 11 | mg | 12 | mg |
| Docosahexaenoic acid | 6.4 | mg | 6.6 | mg |
| Palmitic acid | 580 | mg | 566 | mg |
| sn-2 palmitic acid | 67 | mg | 202 | mg |
| Milk phospholipids | — | | 55 | mg |
| Volume-based mode diameter | 0.5 | μm | 5.6 | μm |
| lipid globules | | | | |
| Volume % of lipid globules | <10 | vol. % | >45 | vol. % |
| with a diameter between 2 | | | | |
| and 12 μm | | | | |

| | | |
|---|---|---|
| Coated with phospholipids | No | Yes |

### Results

The average age at baseline (Visit 1) was 9 days in both randomised groups. Most randomised subjects were Caucasian (90.59%). Birth weight ranged from 2335 to 4180 grams in both randomized groups. The mean gestational age was 39.4 weeks in both groups. The formulas were to be taken until the age of 17 weeks.

During the study (within the first 19 weeks after birth), a lower occurrence of dry skin was observed in the TEST group compared to the CONTROL group, see Table 2. The difference in the number of subjects who did and who did not have one or more times dry skin, comparing TEST and CONTROL group, was statistically significant different (p=0.024, Barnard's test). There was no difference in the number of subjects who did and did not have one or more times dry skin, comparing TEST and Reference (p=0.517, Barnard's test). Statistical analysis was performed using SAS® (SAS Enterprise Guide 4.3 or higher) for Windows (SAS Institute Inc., Cary, N.C.).

TABLE 2
Occurrences of dry skin during the study

| | TEST | CONTROL | Reference |
|---|---|---|---|
| | (n = 112) | (n = 103) | (n = 88) |
| Occurrence of dry skin | 1 | 9 | 0 |
| (no. of events) | | | |
| No. of subjects with | 1 | 7 | 0 |
| one or more events of | | | |
| dry skin | | | |
| % of subjects with one | 0.9% | 6.8% | 0% |
| or more events of | | | |
| dry skin | | | |

1. A non-therapeutic method of preventing dry skin in an infant, comprising feeding said infant a nutritional composition, selected from infant formula, follow on formula and growing up milk, wherein said nutritional composition comprises lipid, protein and digestible carbohydrates, wherein the lipid is in the form of lipid globules, wherein
a. the lipid globules have a mode diameter based on volume of at least 1.0 μm; and
b. at least 45 vol. % of the lipid globules has a diameter of 2 to 12 μm, and
c. the lipid comprises at least 0.5 wt. % phospholipid based on total lipid and the lipid globules have a coating comprising phospholipid,
and wherein the nutritional composition is not human milk and the content of palmitic acid is at least 10 wt. % based on total fatty acids in the total lipid of the nutritional composition and at least 15 wt. % of the palmitic acid, based on total palmitic acid, is in the sn-2 position of a triglyceride.

2. (canceled)

3. The non-therapeutic method according to claim 1, wherein the phospholipid comprises at least 5 wt. % sphingomyelin based on total phospholipid.

4. The non-therapeutic method according to claim 1, wherein the content of palmitic acid is at least 10 wt. % based on total fatty acids in the total lipid of the nutritional composition and at least 25 wt. % of the palmitic acid, based on total palmitic acid, is in the sn-2 position of a triglyceride.

5. (canceled)

6. (canceled)

7. The non-therapeutic method according to claim 1, wherein the lipid comprises at least 0.5 wt. % alpha-linolenic acid based on total fatty acids and at least 5 wt. % linoleic acid based on total fatty acids.

8. The non-therapeutic method according to claim 1, wherein the lipid comprises at least 0.05 wt. % docosahexaenoic acid based on total fatty acids.

9. The non-therapeutic method according to claim 1, wherein the lipid comprises
a. 30 to 90 wt. % vegetable fat based on total lipid, and
b. 10 to 70 wt. % based on total lipid of mammalian milk fat, preferably wherein the mammalian milk fat is derived from the group consisting of butter, butter fat, butter oil, and anhydrous milk fat.

10. The non-therapeutic method according to claim 1, wherein the composition comprises 0.25 to 20 wt. % non-digestible oligosaccharides based on dry weight of the composition.

11. The non-therapeutic method according to claim 10, wherein the non-digestible oligosaccharides comprise galacto-oligosaccharides and/or fructo-oligosaccharides.

12. The non-therapeutic method according to claim 1, wherein the nutritional composition is infant formula or follow on formula, preferably infant formula.

13. The non-therapeutic method according to claim 12, wherein the infant formula or follow on formula comprises 1.8 to 3.5 g protein/100 kcal, 4 to 6 g lipid/100 kcal, 5 to 20 g digestible carbohydrates/100 kcal.

14. The non-therapeutic method according to claim 12, wherein the infant formula or follow-on formula, when ready to drink, comprises 60 to 70 kcal per 100 ml.

15. Use of a nutritional composition, selected from infant formula, follow on formula and growing up milk, which comprises lipid, protein and digestible carbohydrates, wherein the lipid is in the form of lipid globules, wherein
a. the lipid globules have a mode diameter based on volume of at least 1.0 μm; and
b. at least 45 vol. % of the lipid globules has a diameter of 2 to 12 μm, and
c. the lipid comprises at least 0.5 wt. % phospholipid based on total lipid and the lipid globules have a coating comprising phospholipid,
and which is not human milk, for prevention of dry skin in an infant and the content of palmitic acid is at least 10 wt. % based on total fatty acids in the total lipid of the nutritional composition and at least 15 wt. % of the palmitic acid, based on total palmitic acid, is in the sn-2 position of a triglyceride.
